(19) United States
(12) Patent Application Publication
(10) Pub. No.: US 2023/0307114 A1
(43) Pub. Date: Sep. 28, 2023

(54) MEDICAL INFORMATION PROCESSING APPARATUS, MEDICAL INFORMATION PROCESSING METHOD, AND STORAGE MEDIUM

(71) Applicant: CANON MEDICAL SYSTEMS CORPORATION, Otawara-shi (JP)

(72) Inventor: Yohei KAMINAGA, Otawara (JP)

(73) Assignee: CANON MEDICAL SYSTEMS CORPORATION, Otawara-shi (JP)

(21) Appl. No.: 18/188,613

(22) Filed: Mar. 23, 2023

(30) Foreign Application Priority Data
Mar. 25, 2022 (JP) 2022-050672

(51) Int. Cl. G16H 20/40; G16H 50/50

(52) U.S. Cl. CPC G16H 20/40; G16H 50/50

(57) ABSTRACT

A medical information processing apparatus according to an embodiment includes processing circuitry. The processing circuitry estimates a prognosis of a treatment target on the basis of treatment conditions to be applied to the treatment target. The processing circuitry outputs the estimated prognosis of the treatment target via an output interface.

### CROSS-REFERENCE TO RELATED APPLICATION

The present application claims priority based on Japanese Patent Application No. 2022-050672, filed Mar. 25, 2022, the content of which is incorporated herein by reference.

### FIELD

Embodiments disclosed in the present specification and drawings relate to a medical information processing apparatus, a medical information processing method, and a storage medium.

### BACKGROUND

Automation of X-ray irradiation methods and complicated dose calculations has progressed, and many functions of assisting in dose planning to obtain maximum effects of treatment have emerged. On the other hand, information provided by a treatment planning device is specialized for information on treatment methods, and thus information on risks associated with treatment is limited.

While automation of complicated treatment planning has progressed, it relies on the experiences and knowledge of doctors to consider what results (adverse events) will occur with the calculated treatment methods.

### BRIEF DESCRIPTION OF THE DRAWINGS

FIG. 1 is a diagram showing a configuration example of a medical information processing apparatus in a first embodiment.

FIG. 2 is a diagram showing an example of a risk table.

FIG. 3 is a diagram showing an example of a risk table.

FIG. 4 is a diagram showing an example of structured medical procedure information (patient journey).

FIG. 5 is a flowchart showing a flow of a series of processing of processing circuitry according to the first embodiment.

FIG. 6 is a diagram showing an example of a screen displayed on a display.

FIG. 7 is a diagram showing an example of a screen displayed on the display.

FIG. 8 is a diagram showing an example of a screen displayed on the display.

FIG. 9 is a diagram showing an example of a screen displayed on the display.

FIG. 10 is a diagram showing an example of a screen displayed on the display.

FIG. 11 is a diagram showing an example of a screen displayed on the display.

FIG. 12 is a flowchart showing a flow of a series of processing of processing circuitry according to a second embodiment.

FIG. 13 is a diagram schematically showing a machine learning model.

### DETAILED DESCRIPTION

A medical information processing apparatus, a medical information processing method, and a storage medium according to embodiments will be described below with reference to the drawings. A medical information processing apparatus according to an embodiment includes an estimation unit and an output control unit. The estimation unit estimates a prognosis of a treatment target on the basis of treatment conditions applied to the treatment target. The output control unit outputs the estimated prognosis of the treatment target via an output interface. With such a configuration, a prognosis of a patient can be estimated with high accuracy. As a result, a risk of adverse events that may occur in a patient can be appropriately evaluated.

### First Embodiment

### Configuration of Medical Information Processing Apparatus

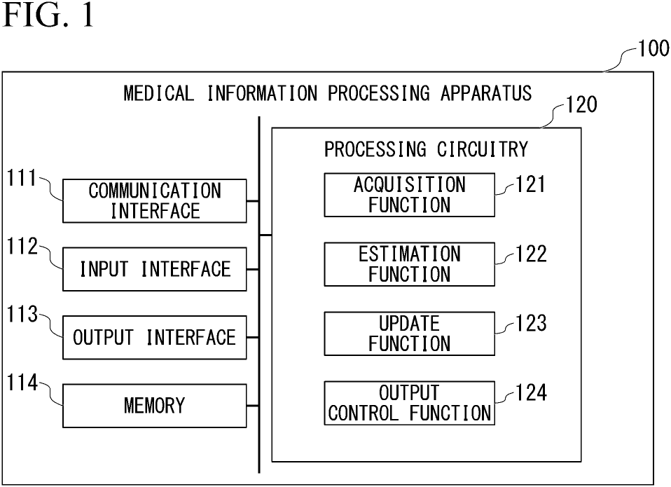

FIG. 1 is a diagram showing a configuration example of a medical information processing apparatus 100 in a first embodiment. The medical information processing apparatus 100 includes, for example, a communication interface 111, an input interface 112, an output interface 113, a memory 114, and processing circuitry 120.

The communication interface 111 communicates with external devices via a communication network NW. The communication network NW may mean a general information communication network using telecommunication technology. For example, the communication network NW includes a telephone communication network, an optical fiber communication network, a cable communication network, a satellite communication network, and the like in addition to a wireless/wired local area network (LAN) such as a hospital backbone LAN, and the Internet network. The communication interface 111 includes, for example, a network interface card (NIC), an antenna for wireless communication, and the like.

The input interface 112 receives various input operations from an operator, converts the received input operations into electrical signals, and outputs the electrical signals to the processing circuitry 120. For example, the input interface 112 includes a mouse, a keyboard, a trackball, a switch, a button, a joystick, a touch panel, and the like. The input interface 112 may be, for example, a user interface that receives voice input, such as a microphone. When the input interface 112 is a touch panel, the input interface 112 may also have a display function of a display 113a included in the output interface 113, which will be described later.

The input interface 112 in this specification is not limited to one including physical operation components such as a mouse and a keyboard. For example, examples of the input interface 112 also include an electrical signal processing circuit that receives an electrical signal corresponding to an input operation from an external input device provided separately from the apparatus and outputs the electrical signal to a control circuit.

The output interface 113 includes, for example, the display 113a, a speaker 113b, and the like. The display 113a displays various types of information. For example, the display 113a displays an image generated by the processing circuitry 120, a graphical user interface (GUI) for receiving various input operations from the operator, and the like. For example, the display 113a is a liquid crystal display (LCD), a cathode ray tube (CRT) display, an organic electroluminescence (EL) display, or the like. The speaker 113b outputs information input from the processing circuitry 120 as sound.

The memory 114 is realized by, for example, a semiconductor memory element such as a random-access memory (RAM) and a flash memory, a hard disk, or an optical disc. These non-transitory storage media may be realized by other storage devices such as a network attached storage (NAS) and an external storage server device connected via the communication network NW. The memory 114 may also include non-transitory storage media such as a read-only memory (ROM) and a register.

The memory 114 stores risk tables, a plurality of structured medical procedure information, electronic medical records, and the like in addition to programs executed by a hardware processor. A risk table is an example of “reference information” and structured medical procedure information is an example of “second reference information.”

A risk table is table data in which each of a plurality of treatment conditions to be referred to is associated with adverse events that may occur when each of treatment conditions is applied, a degree of side effects (side reactions) caused by the adverse events, and the like. For example, when cancer treatment is assumed, treatment conditions include various conditions such as an irradiation range of radiation such as X-rays and electron beams, an irradiation site, and a dose. A treatment condition on a risk table is an example of a “reference treatment condition.”

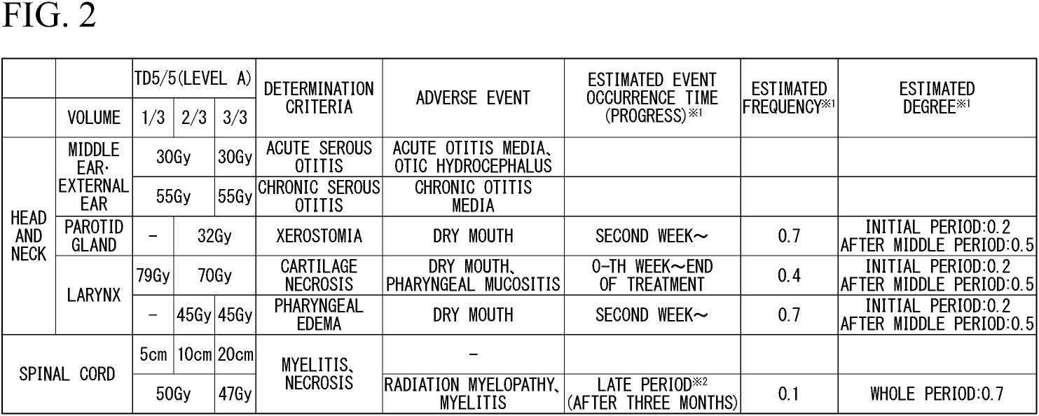

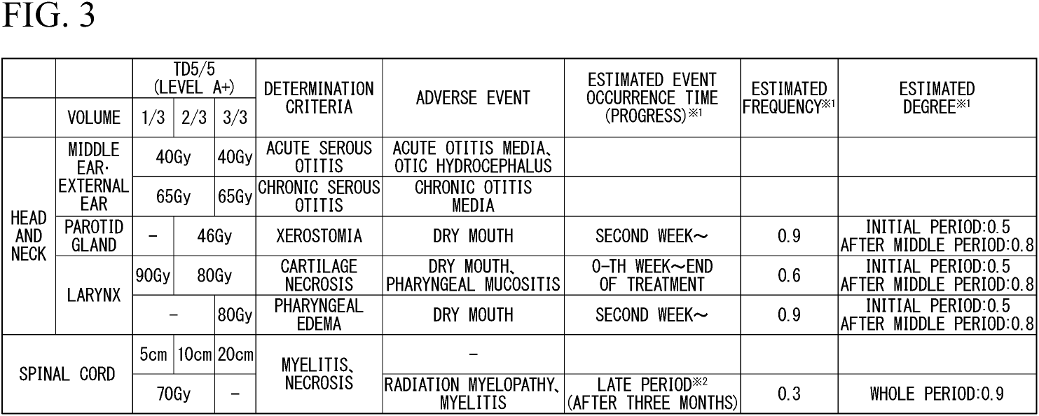

FIG. 2 and FIG. 3 are diagrams showing examples of risk tables. FIG. 2 shows a risk table in the case of a radiation tolerance criterion of TD5/5. TD5/5 is a total dose that produces side effects or disability in 5% of patients over 5 years in simple split irradiation of 2 Gy per day. TD5/5 is a generally accepted dose. The tolerance criterion of TD5/5 will also be referred to as “level A” in the following description. On the other hand, FIG. 3 shows a risk table in the case of a radiation tolerance criterion of TD50/5. TD50/5 is a total dose that produces side effects or disability in 50% of patients over 5 years in simple split irradiation of 2 Gy per day. TD50/5 is a generally unacceptable dose. The tolerance criterion of TD50/5 will also be referred to as “level A+” in the following description.

As shown in each figure, a risk table is present for each radiation tolerance criterion, such as TD5/5 and TD50/5. In the risk table of each tolerance criterion, (1) a tolerable dose, the number of times of irradiation, a volume, and the like are associated, and (2) an adverse event according to treatment conditions (treatment details), timing of occurrence of the adverse event, the frequency of occurrence of the adverse event, and symptom severity of the adverse event are associated for each site such as the head and neck or spinal cord. The risk table is not limited to TD5/5 and TD50/5 and may be present for other tolerance criteria.

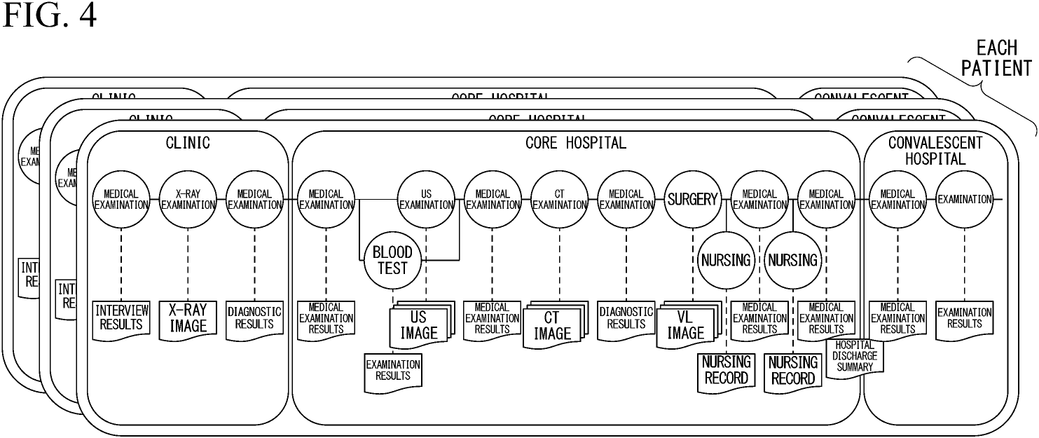

FIG. 4 is a diagram showing an example of structured medical procedure information. The structured medical procedure information is also called a patient journey. Therefore, the structured medical procedure information may be referred to as the patient journey in the following explanation. Structured medical procedure information (patient journey) is information that represents the whole picture of past, present, and/or future medical procedures of each of a plurality of patients. Specifically, structured medical procedure information(patient journey) is information which is structured by medical procedures applied to each patient in the past, medical procedures being currently applied to each patient, or medical procedures to be applied to each patient in the future being associated with patient information of each patient and structured. Patient information is information on a patient generated by medical procedures and includes, for example, interview results, diagnostic results, examination results, nursing records, medical images (X-ray images, CT images, and the like), and the like of the patient. Adverse circumstances recognized by diagnostic results, examination results, nursing records, and the like are reflected in the risk table.

Referring back to FIG. 1, the processing circuitry 120 includes, for example, an acquisition function 121, an estimation function 122, an update function 123, and an output control function 124. The processing circuitry 120 realizes these functions by, for example, a hardware processor (computer) executing a program stored in the memory 114 (storage circuit). The estimation function 122 is an example of an “estimation unit” and the output control function 124 is an example of an “output control unit.”

The hardware processor in the processing circuitry 120 is, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). The program may be configured to be directly embedded in the circuit of the hardware processor instead of being stored in the memory 114. In this case, the hardware processor realizes the functions by reading and executing the program embedded in the circuit. The aforementioned program may be stored in the memory 114 in advance, or may be stored in a non-transitory storage medium such as a DVD or a CD-ROM and installed in the memory 114 from the non-transitory storage medium when the non-transitory storage medium is set in a drive device (not shown) of the user interface 10. The hardware processor is not limited to being configured as a single circuit, and may be configured as one hardware processor by combining a plurality of independent circuits to realize each function. Further, a plurality of components may be integrated into one hardware processor to realize each function.

### Processing Flow of Medical Information Processing Apparatus

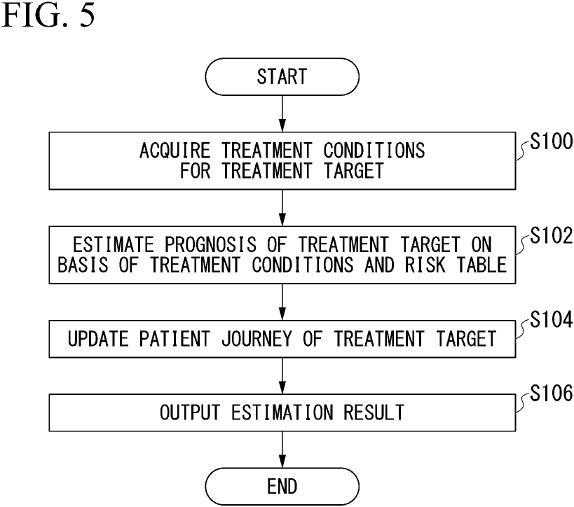

Hereinafter, a series of processing performed by the processing circuitry 120 of the medical information processing apparatus 100 will be described with reference to a flowchart. FIG. 5 is a flowchart showing a flow of a series of processing of the processing circuitry 120 according to the first embodiment. For example, processing of this flowchart may be executed sequentially as a treatment plan progresses, and more specifically, may be executed at intervals such as first to fourth weeks or fifth to sixth weeks.

First, the acquisition function 121 acquires treatment conditions to be applied to a patient to be treated (hereinafter referred to as a treatment target) (step S100). The treatment conditions include, for example, the irradiation site of radiation, the number of times of irradiation, the volume, the dose, and the like.

For example, the acquisition function 121 may acquire the treatment conditions for the treatment target from a radiation treatment planning system (RTPS), which is an external apparatus, via the communication interface 111. Further, if the doctor of the treatment target or the like inputs the treatment conditions for the treatment target to the input interface 112, the acquisition function 121 may acquire the treatment conditions of the treatment target from the input interface 112.

Next, the estimation function 122 estimates a prognosis of the treatment target on the basis of the treatment conditions acquired by the acquisition function 121 and risk tables stored in the memory 114 (step S102).

The estimation function 122 sets levels for the treatment conditions acquired by the acquisition function 121 at the time of estimating the prognosis of the treatment target. Specifically, the estimation function 122 determines the treatment conditions as “level A+” if the dose of radiation to be radiated to the treatment target exceeds TD50/5 and determines the treatment conditions as “level A” if the dose of radiation is TD50/5 or less and exceeds TD5/5 when the treatment conditions acquired by the acquisition function 121 have been applied to the treatment target. Furthermore, the estimation function 122 may determine the treatment conditions as “level B” if the dose of radiation is TD5/5 or less. Such level setting is merely an example and can be arbitrarily changed.

After setting levels for the treatment conditions, the estimation function 122 selects a risk table according to the level of the treatment conditions from among a plurality of risk tables prepared for each tolerance criterion. The estimation function 122 extracts records that match the treatment conditions for the treatment target from the selected risk table and estimates the probability of some or all (typically all) of adverse events included in the records, and the timings, frequency, and symptom severities thereof as a prognosis of the treatment target.

If the treatment conditions for the treatment target are determined to be “level A” and an irradiation site is a “parotid gland,” the estimation function 122 estimates that “dry mouth” will occur as an adverse event when the treatment conditions are applied and further estimates that the “dry mouth” will develop from the second week, the frequency will be about 0.7, the severity of the symptom will be about 0.2 in the early stage and about 0.5 in the middle period and later.

Next, the update function 123 selects structured medical procedure information (patient journey) of the treatment target from among a plurality of structured medical procedure information (patient journeys) of respective patients stored in memory 114 and updates the structured medical procedure information (patient journey) of the treatment target using the prognosis estimated by estimation function 122 (step S104).

For example, the update function 123 updates the structured medical procedure information (patient journey) of the treatment target by associating the estimated prognosis (timings, frequency, and/or severities of symptoms of adverse events) with a medical procedure being currently applied to the treatment target or a medical procedure to be applied in the future.

Next, the output control function 124 outputs the result of estimation performed by the estimation function 122, that is, the prognosis of the treatment target via the output interface 113 (step S106).

For example, the output control function 124 may cause the display 113a of the output interface 113 to display the prognosis of the treatment target. Accordingly, processing of this flowchart ends.

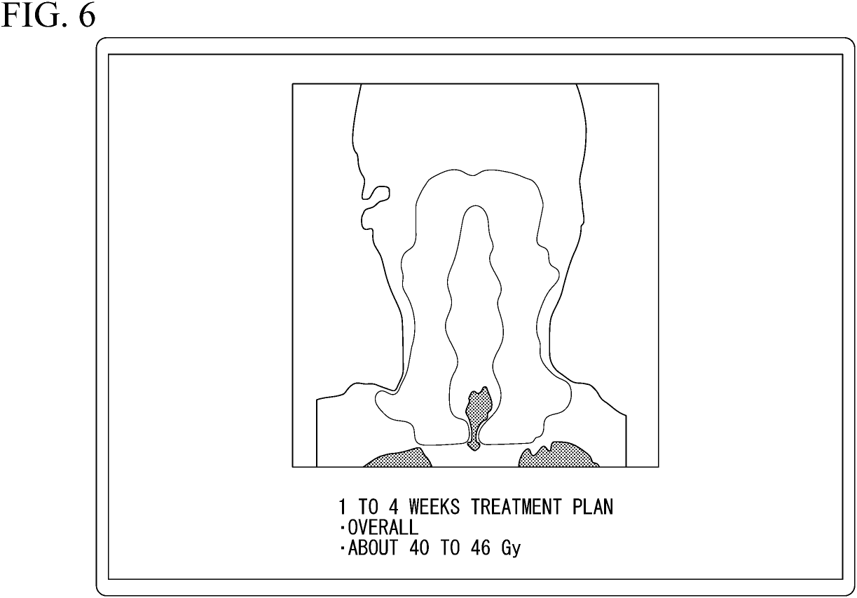

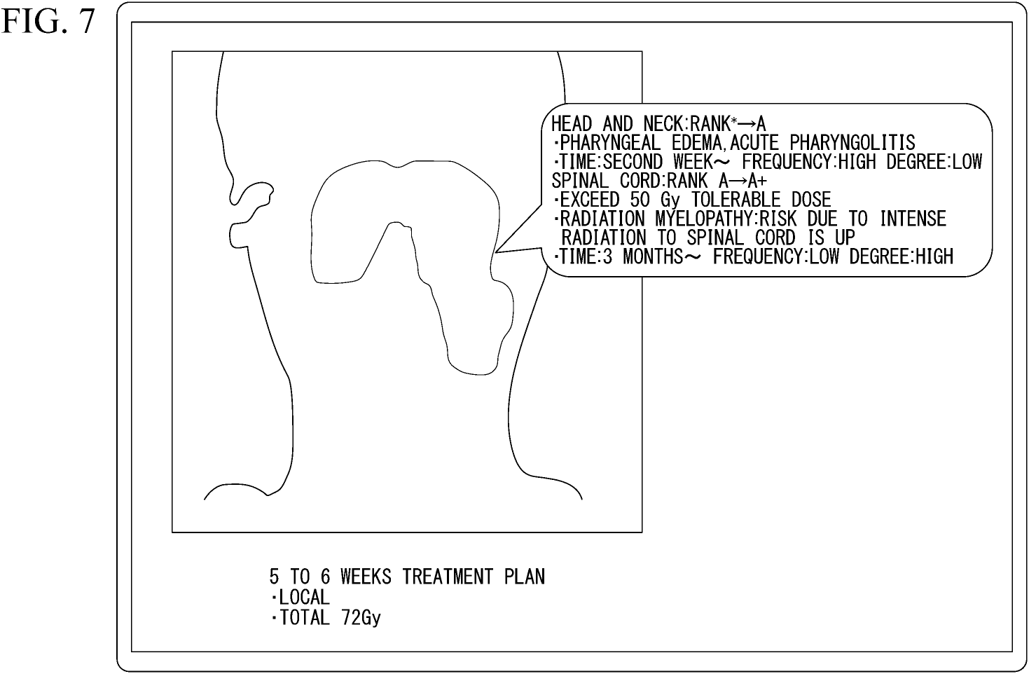

FIG. 6 to FIG. 11 are diagrams showing examples of screens displayed on the display 113a. FIG. 6 shows an example of a medical image captured under treatment conditions of first to fourth weeks, and FIG. 7 shows an example in which adverse events estimated as a prognosis, and timings, frequencies, and/or symptom severities thereof are superimposed on a medical image captured under treatment conditions of fifth and sixth weeks. For example, the output control function 124 may cause the display 113a to display the medical images of FIG. 6 and FIG. 7 side by side in order to compare changes in medical images (X-ray images and CT images) of the treatment target.

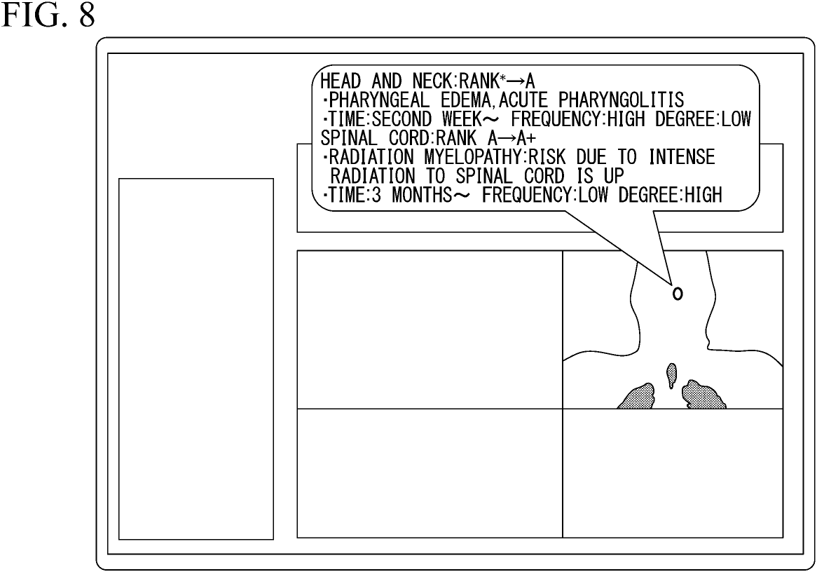

Furthermore, the output control function 124 may display an electronic medical record of the treatment target on which the adverse events estimated as a prognosis, and timings, frequencies, and/or symptom severities thereof have been superimposed, as shown in FIG. 8. In this case, the output control function 124 may display a warning to call attention if the electronic medical record contains notes (adverse events that should not be caused).

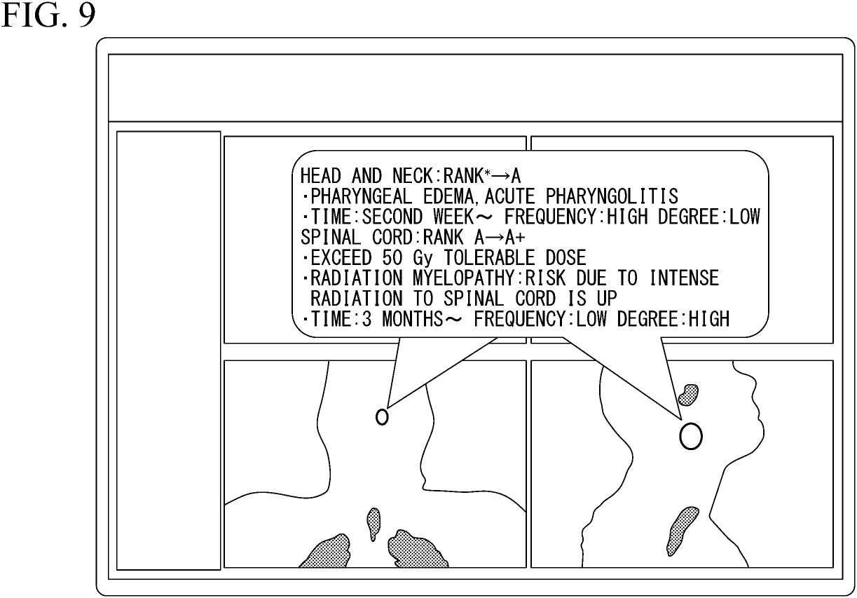

Further, as shown in FIG. 9, the output control function 124 may display a treatment plan for the treatment target on which the adverse events estimated as a prognosis, and timings, frequencies, and/or symptom severities thereof have been superimposed.

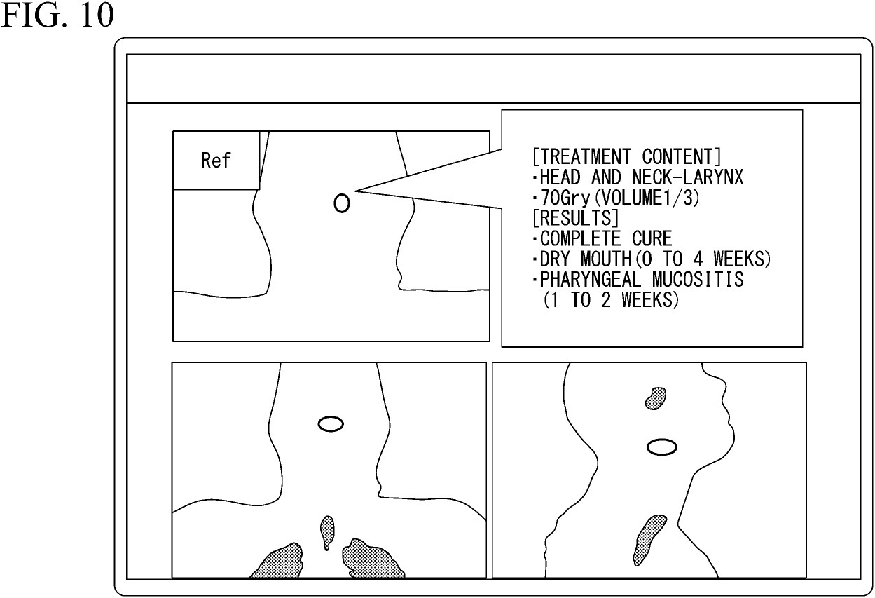

Further, as shown in FIG. 10, the output control function 124 may display a prognosis (Ref in the figure) of another patient whose treatment conditions are similar to those of the treatment target by superimposing the same on the electronic medical record or the treatment plan for the treatment target.

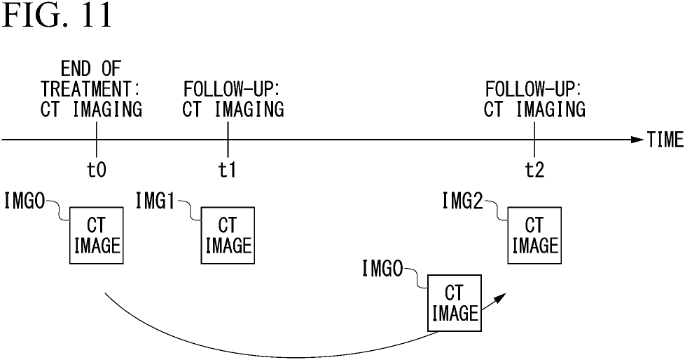

Furthermore, as shown in FIG. 11, the output control function 124 may display a prognosis of the treatment target, estimated at a time t0 in the past, at a time t2 which is later than the time t0. For example, it is assumed that a certain treatment is finished and CT imaging is performed at the time t0. In this case, the output control function 124 superimposes the prognosis estimated at the time t0 on a medical image lMG0 (CT image in the illustrated example) of the treatment target obtained at the time t0 and stores the same in the memory 114. Then, it is assumed that CT imaging is performed at a certain time t2 during the course of treatment follow-up. In this case, the output control function 124 may display the medical image IMG0 on which the prognosis estimated at the time t0 has been superimposed along with a medical image IMG2 of the treatment target obtained at the time t2.

Although the output control function 124 causes the display 113a to display the prognosis of the treatment target in the above-described example, the present disclosure is not limited to this. For example, the output control function 124 may transmit the prognosis of the treatment target to an external device (e.g., radiation treatment planning system, a doctor’s computer, an external display, or the like) via the communication interface 111. For example, when the radiation treatment planning system receives the prognosis of the treatment target from the medical information processing apparatus 100, it may recalculate a treatment plan that does not cause adverse events and transmit the recalculated treatment plan to the medical information processing apparatus 100.

Moreover, although the estimation function 122 estimates a prognosis of the treatment target on the basis of the treatment conditions and the risk table in the above-described example, the present disclosure is not limited thereto. For example, the estimation function 122 may estimate a prognosis of the treatment target on the basis of the structured medical procedure information (patient journey) of the treatment target in addition to the treatment conditions and the risk table. For example, the estimation function 122 may predict adverse events that may occur in the treatment target in the future by combining treatment conditions being currently applied to the treatment target and medical procedures (for example, chemotherapy, examination, surgery, etc.) scheduled to be performed at a certain point in time in the future.

In addition, the treatment conditions may be changed by one function (for example, the estimation function 122 or the like) of the medical information processing apparatus 100 instead of the radiation treatment planning system.

According to the first embodiment described above, the processing circuitry 120 of the medical information processing apparatus 100 acquires treatment conditions to be applied to the treatment target and estimates a prognosis of the treatment target on the basis of the treatment conditions. Then, the processing circuitry 120 outputs the estimated prognosis of the treatment target via the output interface 113. Accordingly, it is possible to appropriately evaluate a risk of adverse events that may occur in the patient by accurately estimating the prognosis of the treatment target.

### Second Embodiment

Hereinafter, a second embodiment will be described. The second embodiment differs from the above-described first embodiment in that the medical information processing apparatus 100 changes treatment conditions. In the following, differences from the first embodiment will be mainly described and a description of points common to the first embodiment will be omitted. In the description of the second embodiment, the same parts as those in the first embodiment are denoted by the same reference numerals.

FIG. 12 is a flowchart showing a flow of a series of processing of the processing circuitry 120 according to the second embodiment. For example, processing of this flowchart may be executed sequentially as a treatment plan progresses, and more specifically, may be executed at intervals such as first to fourth weeks and fifth and sixth weeks.

First, the acquisition function 121 acquires treatment conditions to be applied to a treatment target (step S200).

Next, the estimation function 122 estimates a prognosis of the treatment target on the basis of the treatment conditions acquired by the acquisition function 121 and a risk table stored in the memory 114 (step S202).

Next, the update function 123 selects structured medical procedure information (patient journey) of the treatment target from among the plurality of structured medical procedure information (patient journeys) of respective patients stored in the memory 114 and updates the structured medical procedure information (patient journey) of the treatment target using the prognosis estimated by estimation function 122 (step S204).

Next, the output control function 124 outputs the result of estimation performed by the estimation function 122, that is, the prognosis of the treatment target, via the output interface 113 (step S206).

Next, the estimation function 122 changes (corrects) the treatment conditions applied to the treatment target on the basis of the structured medical procedure information (patient journey) of the treatment target (step S208).

For example, the estimation function 122 may change the treatment conditions such that the irradiation range of radiation radiated to the treatment target is limited. More specifically, the estimation function 122 compares an area that has been irradiated with radiation in the past with an area that is about to be irradiated with radiation this time by referring to past treatment conditions of the treatment target, and when some or all of these areas overlap with each other, changes the treatment conditions such that the irradiation range of radiation is limited to a gross tumor volume (GTV) area. Accordingly, it is possible to reduce a re-irradiation volume because a progress range is limited in recurrence.

In addition, the estimation function 122 may specify a site that should be avoided from being irradiated with radiation and change the treatment conditions such that the site is not irradiated with radiation. More specifically, the estimation function 122 refers to sites that have been irradiated with radiation in the past (hereinafter referred to as exposed sites) from past treatment conditions to change the treatment conditions such that, when a site that is about to be irradiated with radiation this time is the same as a past exposed site, the exposed site is off-target from the re-irradiation range. Assuming that the frequency of adverse events is likely to increase in the middle and subsequent treatments as compared to the initial treatment, normal and high-risk areas can be protected from radiation exposure.

Further, the estimation function 122 may change the treatment conditions such that the dose of radiation is limited. More specifically, the estimation function 122 changes the treatment conditions so as not to exceed a tolerable dose by referring to the dose of past treatment conditions. Assuming that the frequency of adverse events is likely to increase in the middle and subsequent treatments as compared to the initial treatment, the tolerable dose can be strictly controlled.

According to the second embodiment described above, the processing circuitry 120 of the medical information processing apparatus 100 changes some or all of a radiation irradiation range, an irradiation site, and a dose included in treatment conditions to be applied to a treatment target on the basis of the structured medical procedure information (patient journey) of the treatment target. Accordingly, it is possible to assist in reducing a re-irradiation volume, protect normal and high-risk areas from radiation exposure, and strictly control a tolerable dose.

### Third Embodiment

Hereinafter, a third embodiment will be described. The third embodiment differs from the above-described first and second embodiments in that a machine learning model MDL is used to estimate a prognosis of a treatment target. In the following, differences from the first embodiment and the second embodiment will be mainly described, and description of points common to the first embodiment and the second embodiment will be omitted. In the description of the third embodiment, the same parts as those in the first embodiment and the second embodiment are denoted by the same reference numerals.

The memory 114 in the third embodiment stores model information. The model information is information (a program or an algorithm) that defines a machine learning model MDL that has been trained to output a prognosis of a certain patient in response to input of treatment conditions for the patient.

The machine learning model MDL may be implemented by, for example, a neural network. Further, the machine learning model MDL is not limited to neural networks and may be implemented by other models such as a support vector machine, a decision tree, a naive Bayes classifier, and a random forest.

When the machine learning model MDL is implemented by a neural network, the model information includes, for example, coupling information representing how units included in each of an input layer, one or more hidden layers (intermediate layers), and an output layer that constitute the neural network are coupled to each other, weight information representing how many coupling coefficients are provided to data input/output between coupled units, and the like. The coupling information includes, for example, information such as the number of units included in each layer, information for designating the type of a unit that is a coupling destination of each unit, an activation function that realizes each unit, and a gate provided between units in the hidden layers. The activation function that realizes a unit may be, for example, a rectified linear unit (ReLU) function, an exponential linear units (ELU) function, a clipping function, a sigmoid function, a step function, a hyperbolic tangent function, an identity function, or the like. The gate selectively passes or weights data transmitted between units, for example, depending on a value (e.g., 1 or 0) returned by the activation function. A coupling coefficient includes, for example, a weight applied to output data when data is output from a unit in a certain layer to a unit in a deeper layer in a hidden layer of the neural network. Further, the coupling coefficients may also include a bias component unique to each layer, and the like.

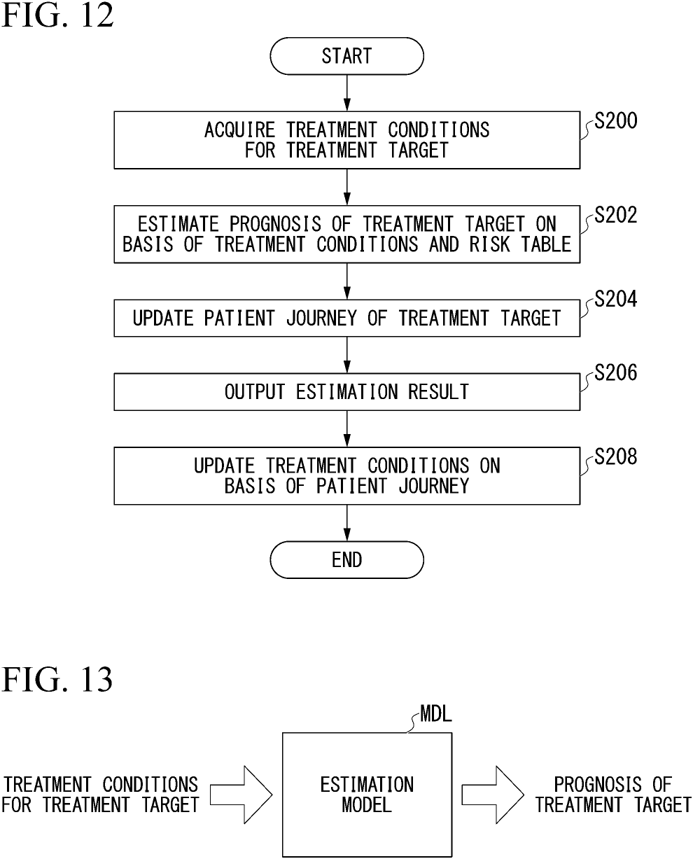

FIG. 13 is a diagram schematically showing the machine learning model MDL. As shown in the figure, the machine learning model MDL is typically trained using a training data set in which adverse events that a patient who is a target for certain training (hereinafter referred to as a training target) has developed, and timings, frequencies, and/or symptom severities of the adverse events are labeled for treatment conditions actually applied to the training target. In other words, the machine learning model MDL is a model trained to output adverse events that a certain training target has developed, and timings, frequencies, and/or symptom severities of the adverse events when treatment conditions for the training target are input. A training target may be the same person as a treatment target or may be a different person. Treatment conditions for a training target included in a training data set is another example of “reference treatment conditions.”

The machine learning model MDL trained in this manner outputs adverse events that a certain training target can develop, and timings, frequencies, and/or symptom severities of the adverse events as an estimation result when treatment conditions for the training target are input thereto.

For example, the estimation function 122 in the third embodiment may estimate a prognosis of a treatment target using the machine learning model MDL instead of or in addition to estimating the prognosis of the treatment target using a risk table. Specifically, the estimation function 122 inputs the treatment conditions acquired by the acquisition function 121 into the pre-trained machine learning model MDL. The machine learning model MDL outputs adverse events that the treatment target can develop, and timings, frequencies, and/or symptom severities of the adverse events in response to the input of the treatment conditions. In response to this, the estimation function 122 estimates the output result of the machine learning model MDL as a prognosis of the treatment target.

According to the third embodiment described above, the processing circuitry 120 of the medical information processing apparatus 100 estimates a prognosis of a treatment target from treatment conditions for the treatment target using the machine learning model MDL trained on the basis of a training data set in which adverse events that a training target has developed, and timings, frequencies, and/or symptom severities of the adverse events are labeled for treatment conditions for the training target. Accordingly, it is possible to accurately estimate the prognosis of the treatment target as in the above-described embodiments, and as a result, it is possible to appropriately evaluate the risk of adverse events that may occur in the patient.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical information processing apparatus comprising processing circuitry configured to:
estimate a prognosis of a treatment target on the basis of treatment conditions to be applied to the treatment target; and
output the estimated prognosis of the treatment target via an output interface.

2. The medical information processing apparatus according to claim 1, wherein the processing circuitry estimates adverse events that are able to occur in the treatment target as the prognosis of the treatment target on the basis of reference information in which adverse events that are able to occur when each reference treatment condition is applied are associated with each of a plurality of reference treatment conditions, and the treatment conditions to be applied to the treatment target.

3. The medical information processing apparatus according to claim 2, wherein, in the reference information, some or all of timings of occurrence of the adverse events, frequencies of occurrence of the adverse events, and severities of symptoms of the adverse events are further associated with the reference treatment conditions, and
the processing circuitry further estimates some or all of timings, frequencies, and symptom severities of the adverse events that are able to occur in the treatment target as the prognosis of the treatment target on the basis of the reference information and the treatment conditions to be applied to the treatment target.

4. The medical information processing apparatus according to claim 3, wherein, in the reference information, the timings, the frequencies, or the severities of the symptoms are further determined for each tolerance criterion of the reference treatment conditions, and
the processing circuitry estimates some or all of the timings, frequencies, and symptom severities of the adverse events that are able to occur in the treatment target according to tolerance criteria of the treatment conditions to be applied to the treatment target.

5. The medical information processing apparatus according to claim 1, wherein the processing circuitry estimates the adverse events that are able to occur in the treatment target as the prognosis of the treatment target by inputting the treatment conditions to be applied to the treatment target into a machine learning model, and
the machine learning model is trained on the basis of a training data set in which adverse events that are able to occur when the reference treatment conditions are applied are associated with the reference treatment conditions.

6. The medical information processing apparatus according to claim 2, wherein the processing circuitry estimates the prognosis of the treatment target further on the basis of second reference information in which a medical procedure applied to each patent in the past, a medical procedure currently being applied, or a medical procedure to be applied in the future is associated with patient information of each of a plurality of patients including the treatment target in addition to the reference information and the treatment conditions to be applied to the treatment target.

7. The medical information processing apparatus according to claim 6, wherein the processing circuitry further changes the treatment conditions to be applied to the treatment target on the basis of the second reference information.

8. The medical information processing apparatus according to claim 7, wherein the treatment conditions are conditions for treating the treatment target by irradiating the treatment target with radiation, and
the processing circuitry changes some or all of an irradiation range, an irradiation site, and a dose of the radiation included in the treatment conditions.

9. The medical information processing apparatus according to claim 1, wherein the output interface includes a display, and
the processing circuitry causes the display to display the estimated prognosis of the treatment target and a medical image of the treatment target together.

10. The medical information processing apparatus according to claim 1, wherein the output interface includes a display, and
the processing circuitry causes the display to display the estimated prognosis of the treatment target and a treatment plan for the treatment target together.

11. The medical information processing apparatus according to claim 1, wherein the output interface includes a display, and
the processing circuitry causes the display to display the estimated prognosis of the treatment target and electronic medical records of the treatment target together.

12. A medical information processing method executed by a computer, comprising:
estimating a prognosis of a treatment target on the basis of treatment conditions to be applied to the treatment target; and
outputting the estimated prognosis of the treatment target via an output interface.

13. A computer-readable non-transitory storage medium storing a program for being executed by a computer and comprising:
estimating a prognosis of a treatment target on the basis of treatment conditions to be applied to the treatment target; and
outputting the estimated prognosis of the treatment target via an output interface.
